(19) United States
(12) Patent Application Publication
(10) Pub. No.: US 2023/0274820 A1
(43) Pub. Date: Aug. 31, 2023

(54) SENSOR-BASED SYSTEM AND METHOD FOR DISPATCH AND ALLOCATION OF MEDICAL DEVICES AND THE LIKE

(71) Applicant: ZEBRA TECHNOLOGIES CORPORATION, Lincolnshire, IL (US)

(72) Inventors: Miroslav Trajkovic, Setauket, NY (US); Stuart Peter Hubbard, Loughborough (GB)

(21) Appl. No.: 17/683,012

(22) Filed: Feb. 28, 2022

(51) Int. Cl. G16H 40/20; G16H 40/40; G16H 20/10; G16H 10/60

(52) U.S. Cl. CPC G16H 40/20; G16H 10/60; G16H 20/10; G16H 40/40

(57) ABSTRACT

A system and method for resource and task allocation are disclosed herein. An example system includes a tracking subsystem configured to track locations and availability of resources; a data acquisition subsystem configured to obtain current status attributes for a patient; a condition detection engine configured to determine, based on the current status attributes and historical medical data, a predicted condition for the patient; a response engine configured to determine, based on the predicted condition, the historical medical data, and a medical history for the patient, a response plan including a resource type to respond to the predicted condition; an allocation engine configured to: obtain the response plan from the response engine; obtain, from the tracking subsystem, tracking data for each resource of the resource type; allocate a resource to be administered to the patient based on the current status attributes of the patient and the tracking data of the resource.

### BACKGROUND

When a patient is admitted to a medical facility, it is important to ensure that proper care is provided, by diagnosing the condition, monitoring and analyzing vital signs, and ensuring proper resources are made available to treat the condition. Resources may be scattered throughout the facility and inefficiencies in identifying an appropriate resource to use and its availability may cause time delays resulting in lower quality care.

### SUMMARY

In an embodiment, the present invention is a sensor-based system for dispatch and allocation of medical devices and the like, the system including: a tracking subsystem configured to track locations and availability of resources, the tracking subsystem including one or more tracking sensors to track respective locations of each resource; a data acquisition subsystem configured to obtain current status attributes for a patient, the data acquisition subsystem including: a direct sensor configured to detect a measurable current status attribute of the patient; an indirect sensor configured to capture raw data representing the patient; and a processor configured to analyze the raw data captured by the indirect sensor and extract one or more derived current status attributes of the patient; a condition detection engine configured to determine, based on the current status attributes and historical medical data, a predicted condition for the patient; a response engine configured to determine, based on the predicted condition, the historical medical data, and a medical history for the patient, a response plan including a resource type to respond to the predicted condition; an allocation engine configured to: obtain the response plan from the response engine; obtain, from the tracking subsystem, tracking data for each resource of the resource type, the tracking data including the respective location obtained from one of the tracking sensors; allocate a resource to be administered to the patient based on the current status attributes of the patient and the tracking data of the resource.

In another embodiment, the present invention is a sensor-based method for dispatch and allocation of medical devices and the like, the method comprising: tracking, via one or more tracking sensors of a tracking subsystem, locations and availability of resources; obtaining current status attributes for a patient including: detecting, via a direct sensor, a measurable current status attribute of the patient; capturing, via an indirect sensor, raw data representing the patient; and analyzing, at a processor, the raw data to extract a current status attribute of the patient; determining, based on the current status attributes and historical medical data, a predicted condition for the patient; determining, based on the predicted condition, the historical medical data, and a medical history for the patient, a response plan including a resource type to respond to the predicted condition; obtaining the response plan from the response engine; obtaining, from the tracking subsystem, tracking data for each resource of the resource type, the tracking data including a respective location obtained from one of the tracking sensors; and allocating a resource to be administered to the patient based on the current status attributes of the patient and the tracking data of the resource.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a schematic diagram of a system for resource and task allocation.

FIG. 2 illustrates a block diagram of the server of the system of FIG. 1.

FIG. 3 illustrates a flowchart of an example method of resource and task allocation in the system of FIG. 1.

FIG. 4 illustrates a flowchart of an example method of allocation at block 425 of the method of FIG. 4.

FIG. 5 illustrates a flowchart of another method of allocation at block 425 of the method of FIG. 4.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

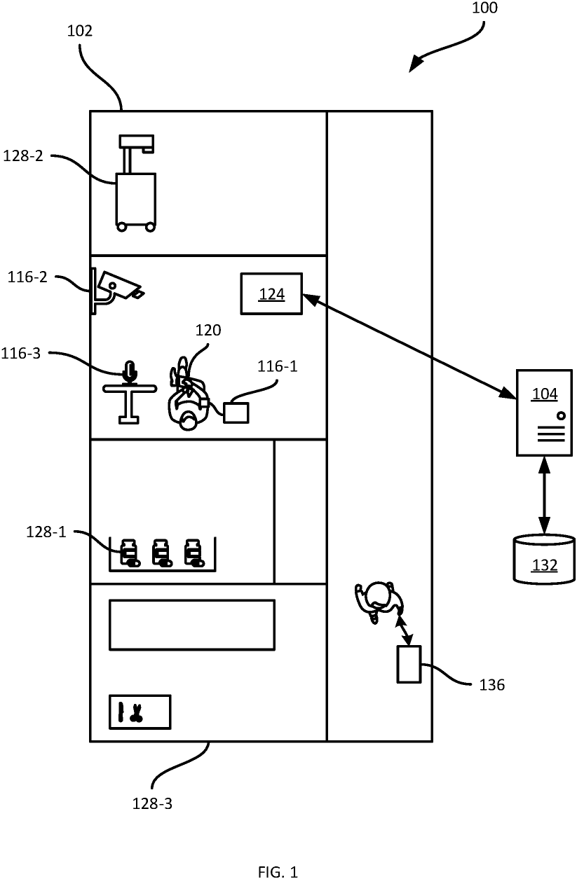

FIG. 1 is a schematic diagram depicting a system 100 for resource allocation for medical triage in accordance with the teachings of this disclosure. The system 100 may be deployed in association with a facility 102, such as a hospital, doctor’s office, or other medical treatment facility. The system 100 includes a server 104 configured to implement the resource allocation for medical triage, a data acquisition subsystem to obtain current status attributes for patients, and a tracking subsystem configured to track locations and availability of various resources in the facility 102. Generally, the system 100 allows for the acquisition of current status attributes for a patient to predict a condition of the patient and determine a potential response plan to triage the patient. The system 100 may then be employed to determine the locations and availability of the resource(s) specified in the response plan and allocate one or more resources to affect the response plan.

The data acquisition subsystem may be deployed in a triage or assessment area of the facility 102 and includes one or more sensors 116. The sensors 116 are generally configured to detect current status attributes for a patient 120. For example, the sensors 116 may include direct sensors, such as a blood pressure sensor 116-1. The direct sensors are generally configured to directly detect a measurable attribute of the patient 120, such as the patient’s heart rate, blood pressure, temperature, or the like. The sensors 116 may further include indirect sensors configured to capture data from which attributes of the patient 120 may be derived. For example, the indirect sensors may include a camera 116-2 or other image sensor, to capture image data representing the patient 120. The image data may then be analyzed to derive visually perceptible attributes of the patient 120, such as a rash or discoloration on the patient’s skin, sweating or shivering of the patient, or the like. In other examples, the indirect sensors may include a microphone 116-3 or other audio sensor, to detect auditory cues from the patient 120. For example, the audio sensors may capture the patient’s verbal description to a medical professional about their symptoms and medical history, a subjective self-assessment, and the like. Other types of direct and/or indirect sensors 116 to detect current status attributes of the patient 120 are also contemplated.

The data acquisition subsystem may further include a processor 124 configured to process the data obtained at the sensors 116 to derive attributes of the patient 120 from the indirect sensors, and to aggregate the derived attributes with the measured attributes from the direct sensors to a set of current status attributes for the patient 120. For example, the processor 124 may be configured to perform video and/or image analytics on the image data captured by the camera 116-2 in order to derive the visually perceptible attributes of the patient 120. The computer vision analytics performed by the processor 124 may also detect behaviors (e.g., minor tremors or the like) captured by the camera 116-2 but which are not visible to the human eye. The processor 124 may additionally be configured to apply natural language processing (NLP) to the audio data captured by the microphone 116-3 to identify and extract keywords which may represent a derived attribute of the patient 120. The audio processing performed by the processor 124 may also detect speech patterns or qualities (e.g., slurring of speech) captured by the microphone 116-3 but which may not otherwise be detectable by the human ear. The processor 124 may reference one or more models and/or artificial intelligence engines to derive the attributes from the image data and the audio data which are relevant as symptoms or indicators for medical triage and diagnosis. In some examples, the processor 124 may be integrated with the server 104. That is, the server 104 may implement the functions of the processor 124 to obtain the derived attributes of the patient 120 and to aggregate the current status attributes of the patient 120.

The tracking subsystem may be deployed throughout the facility 102 and is generally configured to track resources 128 within the facility 102. For example, the resources 128 may include consumable resources, such as medication 128-1, or other consumable products, such as bandages, gauze, syringes, and the like. The resources 128 may also include portable resources, such as a portable x-ray machine 128-2, or other portable machinery or reusable medical equipment the like, which are portable and can be transported between patients and may be used to treat patients or to make further assessments of the patient’s condition. The resources 128 may also include fixed resources, such as an operating room 128-3, or other fixed equipment or spaces, such as a fixed x-ray machine, an operating room, intensive care unit room, or the like, which are fixed in location within the facility 102 and may also be used to treat patients or to make further assessments of the patient’s condition. In some examples, the resources 128 may include resources across multiple physical sites or facilities.

The tracking subsystem may include location tracking sensors or other mechanisms enabling periodic retrieval of the locations of the resources 128 and a repository 132 storing the locations of the resources 128. For example, the tracking subsystem may include wireless emitters deployed throughout the facility, such as wireless access points, beacons (e.g., Bluetooth beacons), radio frequency identification (RFID) readers and the like. The manner of tracking the locations of the resources 128 may vary based on the type of resource 128 being tracked.

For example, the consumable resources may have associated RFID tags or other identifiers, which may be detected by the emitters. In some examples, the tags may be associated with a container or bin holding the consumable resource. The location of the medication 128-1 may then be transmitted by the emitters to the repository 132. The portable resources may include an integrated or supplementary computing device, including a processor, memory, and communications interface enabling the portable resource to determine its location based on signal strength measurements and/or other parameters determined from the signals generated by the emitters. The portable resource may then also transmit its determined location to the repository 132. The fixed resources, being fixed in location within the facility 102 may have a static pre-defined location stored in the repository 132.

In some examples, the tracking subsystem may further include cameras or other sensors configured to recognize and detect the resources 128, such as the portable x-ray machine 128-2, for example, from video streams captured by the cameras.

In some examples, in addition to tracking the locations of resources 128, the tracking subsystem may additionally track the locations of medical personnel within the facility 102. For example, the medical personnel may carry mobile devices 136 which may determine their locations based on signal strength measurements and/or other parameters determined from the signals generated by the emitters. The locations of the medical personnel may then be tracked in the repository 132.

Further, in addition to tracking locations, the tracking subsystem may be configured to track availability via time or stock of the various resources 128. For example, the availability of consumable resources may be tracked by the tracking subsystem based on a stock level of the consumable resource. More particularly, the stock level of regulated resources, such as the medication 128-1, may be tracked quantitatively based on prescriptive issuance of the regulated consumable resource in the repository 132. In other examples, unregulated consumable resources, such as bandages or the like, may include sensors (e.g., weight sensors or the like) associated with a container holding the consumable resource, to determine or derive the remaining stock level. The availability of portable resources, such as the portable x-ray machine 128-2, may be tracked based on a usage schedule for the portable resource. The schedule may define a plurality of appointments including time and location parameters for the portable resource. The time and location parameters may include a target destination, a scheduled time and length of the appointment, and a predicted travel time between consecutive appointments. The schedule for the portable medical equipment may be stored in the repository 132. The availability of the fixed resources, such as the operating room 128-3 may also be tracked based on a usage schedule. The schedule may define a plurality of appointments including time parameters, with the location parameters being fixed due to the fixed location of the fixed resource. The time parameters may include a scheduled time and length of each appointment, as well as a turnover time to prepare the fixed resource (e.g. to clean or sanitize the fixed resource) between consecutive appointments. The schedule for the fixed resource may be stored in the repository 132.

In some examples, some or all of the functionality of the tracking subsystem may be integrated with the server 104. For example, the repository 132 may be stored at the server 104.

The server 104 and the processor 124 may be edge devices, implemented in a server system or other suitable computing system on premises of the facility 102, implemented in a cloud-based system, combinations of the above, and the like.

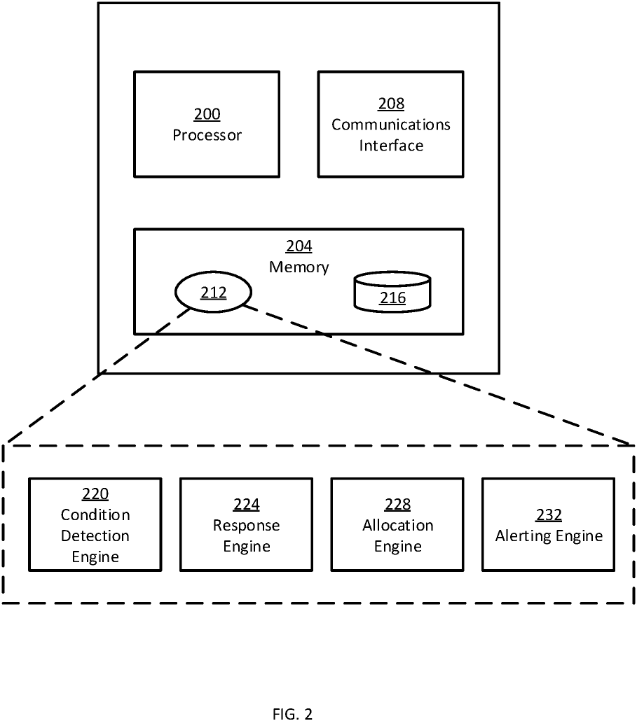

Turning to FIG. 2, certain internal components of the server 104 are illustrated. The server 104 includes a controller, such as a processor 200, such as, for example, one or more microprocessors, controllers, and/or any suitable type of processor. The processor 200 is interconnected with a non-transitory computer readable storage medium, such as a memory 204. The memory 204 includes a combination of volatile memory (e.g., Random Access Memory or RAM) and non-volatile memory (e.g., read only memory or ROM, Electrically Erasable Programmable Read Only memory or EEPROM, flash memory). The processor 200 and the memory 204 may each comprise one or more integrated circuits. The server 104 also includes a communications interface 208 enabling the server 104 to exchange data with other devices and or systems, such as devices of the data acquisition subsystem and the tracking subsystem.

The memory 204 stores computer readable instructions for execution by the processor 200. In particular, the memory 204 stores a resource allocation application 212 which, when executed by the processor 200, configures the processor 200 to triage patients and allocate resources. Those skilled in the art will appreciate that the functionality implemented by the processor 200 via the execution of the application 212 may also be implemented by one or more specially designed hardware and firmware components, such as FPGAs, ASICs, and the like in other embodiments.

The memory 204 further stores a repository 216 containing historical medical data. The historical medical data may include patient identifiers and a medical history for each patient, associations of input attributes (e.g., symptoms or the like) and subsequent assessed conditions determined based on the input attributes, treatment and/or other response options (e.g., further assessments prior to a complete diagnosis) undertaken for each condition assessed, and the like. In other examples, the repository 216 may be stored separately from the server 104 and accessed by the server 104 using the communications interface 208. Additionally, in some examples, the repository 216 may store historical medical data for patients and conditions addressed in the facility 102, as well as other cooperating facilities to allow for a wider database of historical medical data.

The server 104 also includes the communications interface 208 interconnected with the processor 200. The communications interface 208 includes any suitable hardware (e.g., transmitters, receivers, network interface controllers and the like) allowing the server 104 to communicate with other computing devices (e.g., devices of the data acquisition subsystem and the tracking subsystem) via a suitable combination of local and/or wide-area networks. The specific components of the communications interface 208 are selected based on the type(s) of network(s) used by the server 104.

The application 212 implements a condition detection engine 220, a response engine 224, and an allocation engine 228. The condition detection engine 220 is configured to obtain, from the data acquisition subsystem, the current status attributes of the patient 120 and determine a predicted condition for the patient 120 based on the current status attributes. In particular, the predicted condition may be a diagnosis or assessment for the patient 120, including an assessment of a level of urgency of care, or the like. For example, the condition detection engine 220 may implement one or more artificial intelligence algorithms (e.g., neural network, machine learning, etc.) using the historical medical data stored in the repository 216 as a model to predict a condition based on assessed conditions having similar input attributes to the current status attributes of the patient 120.

The response engine 224 is configured to generate a response plan based on the predicted condition. In particular, the response engine 224 may implement one or more artificial intelligence algorithms using the historical medical data stored in the repository 216 as a model to generate a response plan based on response plans applied to patients with a similar patient medical history as the patient 120.

The allocation engine 228 is configured to allocate resources based on the determined response plan. In particular, the allocation engine 228 obtains, from the tracking subsystem, the availability and locations of one or more of the resources 128 specified in the response plan, and allocates the resources 128 to administer the response plan for the patient 120. For example, the allocation engine 228 may select a particular portable resource based on the locations of various types of the same portable resource. The allocation engine 228 may additionally add an appointment to the schedule for the selected portable resource to allocate a time slot in the schedule for use for the generated response plan to attend to the patient 120. In some examples, the allocation engine 228 may additionally allocate one or more tasks to medical personnel, for example by selecting a mobile device 136, as part of the response plan. For example, the task may be to prepare a fixed resource to receive the patient, to retrieve and administer a consumable resource, to retrieve and operate a portable resource, or the like. The allocation engine 228 may be configured to allocate resources across multiple physical sites or facilities 102.

The application 212 may additionally implement an alerting engine 232 configured to cooperate with the condition detection engine 220, the response engine 224, and the allocation engine 228 to generate alerts upon detecting certain criteria. For example, if the level of urgency of the predicted condition exceeds a threshold level, or if the allocation identifies one or more tasks which require review or action by medical personnel, the alerting engine 232 may be configured to generate alerts.

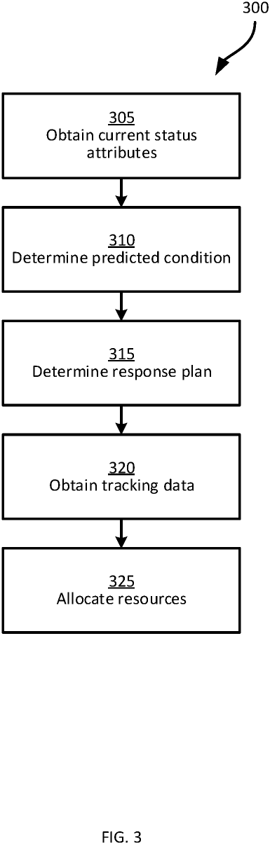

Turning now to FIG. 3, a flowchart of an example method 300 of resource allocation is depicted. The method 300 will be discussed below in conjunction with its performance in the system 100. In other examples, the method 300 may be performed by other suitable devices and/or systems.

At block 305, the data acquisition subsystem obtains current status attributes for the patient 120. The data acquisition subsystem may obtain directly measured attributes, as well as derived attributes. For example, the data acquisition subsystem may obtain various vital signs (e.g., blood pressure, heart rate, temperature, and the like) and other measurable attributes of the patient 120 via the direct sensors. The data acquisition subsystem may further obtain raw image data from the camera 116-2 and apply image processing and/or computer vision analysis at the processor 124 to derive visually perceptible attributes of the patient 120. The data acquisition subsystem may additionally obtain raw audio data from the microphone 116-3 and apply NLP algorithms at the processor 124 to extract keywords as derived attributes of the patient 120.

The data acquisition subsystem may then aggregate the direct attributes and the derived attributes into the current status attributes representing the current status of the patient 120 and transmit the current status attributes to the server 104 for additional processing to triage the patient 120. In other examples, rather than processing the raw image data and raw audio data at the processor 124, the data acquisition subsystem may send the raw image data and the raw audio data to the server 104 to analyze for the derived attributes.

At block 310, the server 104, and in particular the condition detection engine 220, having received the current status attributes of the patient 120, analyzes the current status attributes of the patient 120 to determine a predicted condition for the patient 120. In particular, the condition detection engine 220 may retrieve historical medical data from the repository 132 to use as a model to predict the condition of the patient 120. For example, the condition detection engine 220 may use the current status attributes as an input and retrieve, from the historical medical data, cases having patients with similar input attributes. The condition detection engine 220 may use the assessed conditions from those cases to generate a predicted condition for the patient 120. Additionally, in some examples, the condition detection engine 220 may narrow the cases and corresponding assessed conditions based on the individual medical history of the patient 120 and the patients in the cases. In some examples, the predicted condition generated by the condition detection engine 220 may also include a level of urgency of the condition. For example, if the condition detected suggests that the patient is having a heart attack, the condition detection enginge 220 may return an assessment with a high level of urgency. If the condition detected suggests that the patient has a scratch, particularly a shallow one, the condition detection engine 220 may return an assessment with a low level of urgency. The condition detection engine 220 may cooperate with the alerting engine 232 to generate alerts based on the level of urgency of the condition. For example, upon detection of a predicted condition or a level of urgency satisfying certain predefined criteria (e.g., matching one of a subset of conditions, or having a threshold urgency), the alerting engine 232 may generate and send an alert to one of the mobile devices 136 for attention by a medical personnel. Some or all of the analysis performed by the condition detection engine 220 may be performed by, enhanced, or confirmed using artificial intelligence algorithms to generate the predicted condition.

At block 315, the server 104, and in particular the response engine 224, is configured to determine a response plan based on the predicted condition determined at block 310. In particular, the response engine 224 may retrieve historical medical data from the repository 132 to use as a model to determine a response plan for the patient 120. In particular, the response engine 224 may use the predicted condition generated at block 310 as an input and retrieve, from the historical medical data, cases having patients with an assessed condition matching or medically similar to the predicted condition. The response engine 224 may use the response plans from those cases to develop a response plan for the patient 120. In some examples, the response engine 224 may additionally reference inputs from medical personnel and/or new research developments in conjunction with the historical medical data to determine the response plan. The response engine 224 may additionally analyze the outcome of the cases to develop a response plan. That is, cases from the historical medical data having a more positive outcome (e.g., improvement in the patient’s status attributes and/or recovery from the condition) may be more heavily weighted by the response engine 224 than cases having a negative outcome (e.g., patient death or severe decrease to quality of life) in developing the response plan. Additionally, the response engine 224 may consider the individual medical history of the patient 120 as well as the current status attributes of the patient 120, and cases having patients with a similar medical history and/or similar input attributes and having a positive outcome as a result of the undertaken response plan. Some or all of the analysis performed by the response engine 224 may be performed by, enhanced, or confirmed using artificial intelligence algorithms to generate the response plan.

The response plan generated by the response engine 224 may include various resource types to utilize in response to the predicted condition of the patient 120. For example, the response plan may specify resource types to be used in the alternative (e.g., a fixed x-ray machine versus a portable x-ray machine), and/or resource types to be used in tandem (e.g., pain medication and/or bandages to temporarily address pain and/or wounds experienced by the patient, as well as further treatment and/or assessment via portable or fixed resources to better diagnose and/or more robustly address the patient’s condition). The response plan may additionally specify the amount of each resource type to be used, expressed, for example by a quantity of the consumable resources, a length of appointment of the portable or fixed resources, or the like.

At block 320, the server 104, and in particular the allocation engine 228, is configured to obtain, from the tracking subsystem, tracking data each resource 128 of the resource type(s) specified in the response plan generated at block 315. The tracking data may include the locations and/or availability of the resources 128.

At block 325, the server 104, and in particular the allocation engine 228, allocates resources to effect at least part of the response plan generated at block 315. In particular, allocating a resource may include reserving the resource for administration to the patient 120. In some examples, allocating a resource at block 325 may additionally include allocating one or more tasks to medical personnel via their associated mobile devices 136 to administer the resource to the patient 120. Further, the allocation engine 228 may cooperate with the alerting engine 232 to generate alerts for the medical personnel, or at the locations of the resources 128 to prepare the resources 128 for administration to the patient 120 after allocation. The manner of allocating resource and/or tasks may vary based on the type of resource.

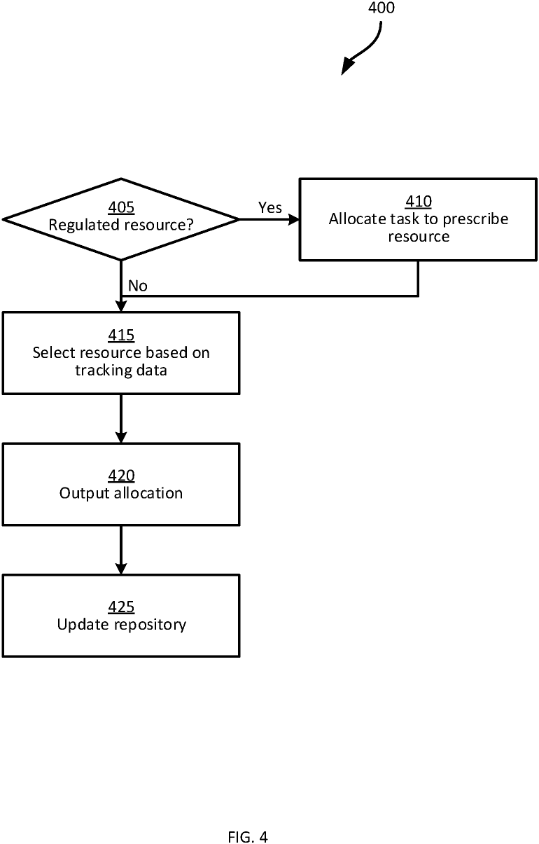

For example, referring to FIG. 4, an example method 400 of allocating a consumable resource is depicted. At block 405, the allocation engine 228 determines whether the consumable resource is a regulated resource. For example, regulated resources may be certain medications or the like which may only be prescribed by a doctor, and which may be prescribed in specific quantities. Unregulated resources may be over-the-counter medications, consumable products, or the like. If, at block 405, the allocation engine 228 determines that the consumable resource is a regulated resource, the allocation engine 228 proceeds to block 410.

At block 410, the allocation engine 228 may allocate a task for a doctor or other qualified professional to prescribe the regulated resource. For example, the allocation engine 228 may allocate a task via the mobile device 136 of the doctor. The allocation engine 228 may be configured to send details of the patient medical history, current status attributes of the patient, the predicted condition determined at block 310, and the recommended response plan determined at block 315 to the mobile device 136, in conjunction with a task to prescribe the regulated resource. The medical professional may then review the details and make the prescription to complete the task. In other examples, the task allocated by the allocation engine 228 may simply be for the doctor or qualified medical professional to go to the patient 120 and make an assessment and/or prescription in person. For example, the allocation engine 228 may select an appropriate task to allocate to the medical professional via the mobile device 136 based on the predicted condition, including the level of urgency of the predicted condition, the type of regulated resource recommended in the response plan, and other relevant factors.

After the regulated resource has been prescribed, the method 400 proceeds to block 415. If, at block 405, the consumable resource specified in the response plan is unregulated, the method 400 may additionally proceed directly to block 415.

At block 415, the allocation engine 228 selects a particular resource to allocate based on the tracking data obtained at block 320. That is, while the response plan may specify a type of resource, at block 415, the allocation engine 228 selects a specific resource of the specified type of resource. For example, the allocation engine 228 may select the specific resource based on the locations and availability of the resources 128, for example by selecting the resource 128 which has a location closest to the patient 120 and/or which has sufficient stock to supply to the patient 120, or the like.

At block 420, the allocation engine 228 outputs the allocation. In some examples, the allocation may be output at the location that the resource selected at block 415 is stored. For example, the allocation may be sent to a pharmacy associated with the particular selected resource to prepare the medication 128-1 to be dispensed. In some examples, the allocation may additionally include one or more task allocations to retrieve and administer the consumable resource. Accordingly, the allocation may be output to one or more of the mobile devices 136 associated with medical personal. In such examples, the allocation output to the selected mobile device 136 may additionally include a location of the allocated resource to facilitate retrieval of the resource and improve efficiency. In some examples, the allocation may additionally or alternately be output at a computing device associated with the triage or assessment area at which the patient 120 is being triaged to allow attending medical personnel to view the allocated resources 128 to administer to the patient 120. In such examples, the allocation output to the computing device may additionally include a location of the allocated resource to facilitate retrieval of the resource or to provide an estimated time of arrival of the resource based on the distance to the patient 120, if a task has been allocated to retrieve the consumable resource.

At block 425, after allocating the consumable resource, the allocation engine 228 may update the repository 132 of the tracking subsystem. For example, the allocation engine 228 may update the repository 132 based on the output allocation at block 420, for example based on the prescription and/or dispensing data generated during allocation of the resource. In other examples, the tracking subsystem may use sensors associated with the consumable resources to detect a change in stock level or location and update the repository 132. The tracking subsystem may be configured to update the stock level in response to an indication from the allocation engine 228 that a consumable resource was allocated and hence that the repository 132 should be updated based on new tracking data.

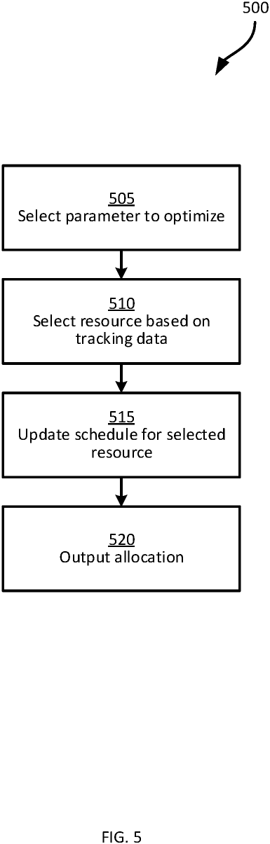

FIG. 5 depicts an example method 500 of allocating a portable resource or a fixed resource. In particular, the allocation engine 228 may allocate portable and fixed resources based on the scheduling availability of the resource, and the location of the resource, particularly with consideration of the amount of time to transport the portable resource to the patient or to transport the patient to the fixed resource. Accordingly, at block 505, the allocation engine 228 may select a parameter to optimize. The allocation engine 228 may select time of administration of the resource or efficiency of transporting the portable resource, or other relevant parameter. The allocation engine 228 may select the parameter based on the predicted condition determined at block 310, or other factors. For example, for a predicted condition with a high level of urgency, the allocation engine 228 may select time of administration of the resource as the parameter to optimize. For less urgent conditions, the allocation engine 228 may select efficiency of transportation of the portable resource or the patient as the parameter to optimize. In some examples, rather than optimizing for a single parameter, the allocation engine 228 may define a weighting of the parameters to optimize.

At block 510, the allocation engine 228 selects a particular resource to allocate. That is, while the response plan may specify a type of resource, at block 510, the allocation engine 228 selects a specific resource of the specified type of resource. The allocation engine 228 may select the specific resource to optimize the parameter selected at block 505. For example, if the patient 120 has a predicted condition with a high level of urgency, the allocation engine 228 may select the first available portable or fixed resource, that is, the resource with the first available appointment time in its schedule, accounting for transportation time from the location of the previous appointment for the portable resource or transportation time from the location of the patient to the fixed resource. For example, this may involve transporting the portable resource from a distant part of the facility 102 or transporting the patient to a distant part of the facility 102. In other examples, if the predicted condition is not urgent and the patient 120 is in stable condition, the allocation engine 228 may optimize transportation of the portable resource and/or the patient. Accordingly, the allocation engine 228 may select a portable resource for which the previous appointment is within a threshold distance of the patient 120, rather than selecting the next available appointment. Similarly, the allocation engine 228 may select a fixed resource which is within a threshold distance of the patient 120.

At block 515, the allocation engine 228 updates the schedule for the resource selected at block 510. The allocation 228 may retrieve, from the response plan generated at block 315, the length of appointment to be booked in the schedule, and add an appointment for the specified length of time. Further, the allocation engine 228 may schedule the appointment with consideration of an amount of time to transport the portable resource from the location of the previous appointment to the patient’s location or to transport the patient to the fixed resource. The allocation engine 228 may propagate the schedule updates to the repository 132.

In some examples, at blocks 510 and 515, rather than adhering to the existing schedule defined in the repository 132, the allocation engine 228 may revise the schedule, including moving existing appointments, to better optimize for time to administer the resource to patients, particularly with regards to the urgency of each patient’s condition, as well as for efficiency of transportation of the portable resources and the patients. In some examples, the allocation engine 228 may additionally consider the schedules of multiple of the same type of portable resource or interchangeable fixed resources to optimize time and transportation efficiency of each resource. The allocation engine 228 may then update the schedules in the repository 132 prior to proceeding to block 520.

At block 520, the allocation engine 228 outputs the allocation. The allocation may be output at the location of the selected resource, for example by sending an indication of the allocation directly to a computing device associated with the portable resource. The allocation may additionally include one or more task allocations to retrieve and administer the portable resource, for example by creating a task for a qualified medical professional to operate the portable x-ray machine 128-2. Such task allocations may be output to one or more of the mobile devices 136 associated with medical personnel. In such examples, the allocation output to the selected mobile device(s) 136 may include a location of the allocated resource to facilitate retrieval of the resource and improve efficiency. The allocation may additionally or alternately be output at a computing device associated with the triage or assessment area at which the patient 120 is being triaged to allow attending medical personnel to view the allocated resources 128 to be administered to the patient 120. The allocation output to the computing device may additionally include a location of the allocated resource to facilitate retrieval of the resource or to provide an estimated time of arrival of the resource based on the distance to the patient 120, if a task has been allocated to retrieve the portable resource. Alternately, the allocation output to the computing device may include a location of the allocated resource to facilitate transportation of the patient 120 to the selected fixed resource.

The above description refers to a block diagram of the accompanying drawings. Alternative implementations of the example represented by the block diagram includes one or more additional or alternative elements, processes and/or devices. Additionally or alternatively, one or more of the example blocks of the diagram may be combined, divided, re-arranged or omitted. Components represented by the blocks of the diagram are implemented by hardware, software, firmware, and/or any combination of hardware, software and/or firmware. In some examples, at least one of the components represented by the blocks is implemented by a logic circuit. As used herein, the term “logic circuit” is expressly defined as a physical device including at least one hardware component configured (e.g., via operation in accordance with a predetermined configuration and/or via execution of stored machine-readable instructions) to control one or more machines and/or perform operations of one or more machines. Examples of a logic circuit include one or more processors, one or more coprocessors, one or more microprocessors, one or more controllers, one or more digital signal processors (DSPs), one or more application specific integrated circuits (ASICs), one or more field programmable gate arrays (FPGAs), one or more microcontroller units (MCUs), one or more hardware accelerators, one or more special-purpose computer chips, and one or more system-on-a-chip (SoC) devices. Some example logic circuits, such as ASICs or FPGAs, are specifically configured hardware for performing operations (e.g., one or more of the operations described herein and represented by the flowcharts of this disclosure, if such are present). Some example logic circuits are hardware that executes machine-readable instructions to perform operations (e.g., one or more of the operations described herein and represented by the flowcharts of this disclosure, if such are present). Some example logic circuits include a combination of specifically configured hardware and hardware that executes machine-readable instructions. The above description refers to various operations described herein and flowcharts that may be appended hereto to illustrate the flow of those operations. Any such flowcharts are representative of example methods disclosed herein. In some examples, the methods represented by the flowcharts implement the apparatus represented by the block diagrams. Alternative implementations of example methods disclosed herein may include additional or alternative operations. Further, operations of alternative implementations of the methods disclosed herein may combined, divided, re-arranged or omitted. In some examples, the operations described herein are implemented by machine-readable instructions (e.g., software and/or firmware) stored on a medium (e.g., a tangible machine-readable medium) for execution by one or more logic circuits (e.g., processor(s)). In some examples, the operations described herein are implemented by one or more configurations of one or more specifically designed logic circuits (e.g., ASIC(s)). In some examples the operations described herein are implemented by a combination of specifically designed logic circuit(s) and machine-readable instructions stored on a medium (e.g., a tangible machine-readable medium) for execution by logic circuit(s).

As used herein, each of the terms “tangible machine-readable medium,” “non-transitory machine-readable medium” and “machine-readable storage device” is expressly defined as a storage medium (e.g., a platter of a hard disk drive, a digital versatile disc, a compact disc, flash memory, read-only memory, random-access memory, etc.) on which machine-readable instructions (e.g., program code in the form of, for example, software and/or firmware) are stored for any suitable duration of time (e.g., permanently, for an extended period of time (e.g., while a program associated with the machine-readable instructions is executing), and/or a short period of time (e.g., while the machine-readable instructions are cached and/or during a buffering process)). Further, as used herein, each of the terms “tangible machine-readable medium,” “non-transitory machine-readable medium” and “machine-readable storage device” is expressly defined to exclude propagating signals. That is, as used in any claim of this patent, none of the terms “tangible machine-readable medium,” “non-transitory machine-readable medium,” and “machine-readable storage device” can be read to be implemented by a propagating signal.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises ...a”, “has ...a”, “includes ...a”, “contains ...a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

1. A sensor-based system for dispatch and allocation of medical devices and the like, the system comprising:
a tracking subsystem configured to track locations and availability of resources, the tracking subsystem including one or more tracking sensors to track respective locations of each resource;
a data acquisition subsystem configured to obtain current status attributes for a patient, the data acquisition subsystem including:
a direct sensor configured to detect a measurable current status attribute of the patient;
an indirect sensor configured to capture raw data representing the patient; and
a processor configured to analyze the raw data captured by the indirect sensor and extract one or more derived current status attributes of the patient;
a condition detection engine configured to determine, based on the current status attributes and historical medical data, a predicted condition for the patient;
a response engine configured to determine, based on the predicted condition, the historical medical data, and a medical history for the patient, a response plan including a resource type to respond to the predicted condition;
an allocation engine configured to:
obtain the response plan from the response engine;
obtain, from the tracking subsystem, tracking data for each resource of the resource type, the tracking data including the respective location obtained from one of the tracking sensors; and
allocate a resource to be administered to the patient based on the current status attributes of the patient and the tracking data of the resource.

2. The system of claim 1, wherein the condition detection engine is further configured to assess a level of urgency of the predicted condition.

3. The system of claim 1, wherein the tracking subsystem comprises a repository configured to store:
a location and stock level of each consumable resource;
a schedule having time parameters and location parameters for each portable resource; and
a fixed location and a schedule having time parameters and location parameters for each fixed resource.

4. The system of claim 1, wherein, to allocate the resource, the allocation engine is configured to allocate a task to a mobile device of a qualified medical professional to prescribe a regulated consumable resource.

5. The system of claim 1, wherein, to allocate the resource, the allocation engine is configured to select the resource having a resource location closest to the patient.

6. The system of claim 1, wherein, to allocate the resource, the allocation engine is configured to select the first available resource for the patient.

7. The system of claim 1, wherein the allocation engine is further configured to allocate a task to retrieve and administer the allocated resource, the resource being a consumable resource or a portable resource.

8. The system of claim 1, wherein the allocation engine is further configured to allocate a task to transport the patient to the allocated resource, the resource being a fixed resource.

9. The system of claim 1, wherein the allocation engine is configured to revise respective schedules of one or more of the resources to optimize administration of the resources.

10. The system of claim 1, further comprising an alerting engine to generate alerts in response to detection of one or more predefined criteria by one of the condition detection engine, the response engine, and the allocation engine.

11. A sensor-based method for dispatch and allocation of medical devices and the like, the method comprising:
tracking, via one or more tracking sensors of a tracking subsystem, locations and availability of resources;
obtaining current status attributes for a patient including:
detecting, via a direct sensor, a measurable current status attribute of the patient;
capturing, via an indirect sensor, raw data representing the patient; and
analyzing, at a processor, the raw data to extract a current status attribute of the patient;
determining, based on the current status attributes and historical medical data, a predicted condition for the patient;
determining, based on the predicted condition, the historical medical data, and a medical history for the patient, a response plan including a resource type to respond to the predicted condition;
obtaining tracking data for each resource of the resource type, the tracking data including a respective location obtained from one of the tracking sensors; and
allocating a resource to be administered to the patient based on the current status attributes of the patient and the tracking data of the resource.

12. The method of claim 11, further comprising assessing a level of urgency of the predicted condition.

13. The method of claim 11, wherein tracking the locations and availability of the resources comprises
storing a location and stock level of each consumable resource;
storing a schedule having time parameters and location parameters for each portable resource; and
storing a fixed location and a schedule having time parameters and location parameters for each fixed resource.

14. The method of claim 11, wherein allocating the resource comprises allocating a task to a mobile device of a qualified medical professional to prescribe a regulated consumable resource.

15. The method of claim 11, wherein allocating the resource comprises selecting the resource having a closest location to the patient.

16. The method of claim 11, wherein allocating the resource comprises selecting the first available resource for the patient.

17. The method of claim 11, further comprising allocating a task to retrieve and administer the allocated resource, the resource being a consumable resource or a portable resource.

18. The method of claim 11, further comprising allocating a task to transport the patient to the allocated resource, the resource being a fixed resource.

19. The method of claim 11, further comprising revising respective schedules of one or more of the resources to optimize administration of the resources.

20. The method of claim 11, further comprising generating alerts in response to detection of one or more predefined criteria.
